(19) United States
(12) Patent Application Publication
(10) Pub. No.: US 2022/0347108 A1
(43) Pub. Date: Nov. 3, 2022

(54) PROCESS FOR PRODUCING A PHARMACEUTICAL FORMULATION COMPRISING CRYSTALLINE AND AMORPHOUS FRACTIONS OF AN ACTIVE SUBSTANCE

(71) Applicant: BAYER AKTIENGESELLSCHAFT, Leverkusen (DE)

(72) Inventors: Michael OSTENDORF, Leverkusen (DE); Werner HOHEISEL, Koeln (DE); Christoph NUEBOLDT, Heessen (DE)

(21) Appl. No.: 17/642,589

(22) PCT Filed: Oct. 5, 2020
(86) PCT No.: PCT/EP2020/077789

§ 371 Date: Mar. 11, 2022

(30) Foreign Application Priority Data
Oct. 10, 2019 (EP) 19202455.2

(51) Int. Cl. A61K 9/50; A61K 9/10; A61K 31/405

(52) U.S. Cl. CPC A61K 9/5026; A61K 9/10; A61K 31/405; A61K 9/5089

(57) ABSTRACT

A process for producing a pharmaceutical formulation comprising the steps of: A) providing particles of a polymer, wherein particles of a pharmaceutical active substance are additionally at least partially embedded in the particles of the polymer; B) heating the particles of the polymer to a predetermined temperature for a predetermined time and C) cooling the particles of the polymer after the predetermined time to a temperature of 18° C. to 24° C., wherein the polymer is at least partially soluble in water and the active substance is at least partially soluble in the polymer.

The particulate pharmaceutical active substance is present in the form of particles having a d₉₀ value in the particle size distribution of ≤1 μm. The predetermined temperature is within a range from 10 K below the glass transition temperature (determined by DSC in accordance with DIN EN ISO 11357-2 at a heating rate of 10 K/min) of the polymer to the melting temperature of the active substance. The total proportion of the active substance in the polymer is greater than the amount of active substance soluble in the polymer at the predetermined temperature.

The invention further relates to a pharmaceutical formulation comprising a particulate pharmaceutical active substance coated with an at least partially water-soluble polymer, to a process for producing a suspension of a pharmaceutical formulation and to a suspension of a pharmaceutical active substance.

The present invention relates to a process for producing a pharmaceutical formulation comprising the steps of: A) providing particles of a polymer, wherein particles of a pharmaceutical active substance are additionally at least partially embedded in the particles of the polymer; B) heating the particles of the polymer to a predetermined temperature for a predetermined time and C) cooling the particles of the polymer after the predetermined time to a temperature of 18° C. to 24° C., wherein the polymer is at least partially soluble in water and the active substance is at least partially soluble in the polymer. The invention further relates to a pharmaceutical formulation comprising a particulate pharmaceutical active substance coated with an at least partially water-soluble polymer, to a process for producing a suspension of a pharmaceutical formulation and to a suspension of a pharmaceutical active substance.

A high rate of dissolution of a pharmaceutical active substance usually results in increased bioavailability or at least in improved bioavailability kinetics. This can be achieved, for example, by increasing the specific surface area of the active substance-particle collective. Thus, active substance nanosuspensions have an appreciably higher rate of dissolution than a micronized suspension. Another method of increasing the rate of dissolution and the solubility is the production of active substance-polymer dispersions in the form of amorphous solids. In this approach, a molecular dispersion of the active substance is produced in a polymer matrix, which provides amorphous stabilization. This system is thermodynamically stable only if the polymer is able to completely dissolve the active substance present. The low solubilities of the active substance in the polymer at room temperature means that usually only a small amount of active substance is soluble in a polymer matrix in a stable manner.

In the article “Stability of nanosuspensions in drug delivery” in the Journal of Controlled Release 172 (2013) 1126-1141, Wang et al. report powders containing redispersible nanoparticles produced by freeze-drying. Besides the usual stabilizers, additional matrix-forming agents were also used here. SDS is used only as an additive for milling.

The review article “Polymeric Amorphous Solid Dispersions: A Review of Amorphization, Crystallization, Stabilization, Solid-State Characterization, and Aqueous Solubilization of Biopharmaceutical Classification System Class II Drugs” by S. Baghel et al. in the Journal of Pharmaceutical Sciences 105, 9, 2016, pp. 2527-2544 describes the production of amorphous solid dispersions. The review does not mention any hybrid systems.

The article “Polymorphism of Indomethacin in Semicrystalline Dispersions: Formation, Transformation, and Segregation” by Van Duong et al. in Mol. Pharmaceutics 2018, 15, 1037-1051 reports semicrystalline polymer-active substance systems in which, starting from an amorphous solid dispersion, different polymorphs can be formed by varying the active substance content. The disadvantage of this method is that there is no controlled growth and thus crystallites of varying size are formed. In addition, crystalline PEG is used. Different polymorphs are moreover formed. The amorphous phase is at the end almost completely crystalline.

The object of the present invention is to provide improved pharmaceutical formulations that permit higher loading of active substance alongside variable release kinetics.

This object is achieved in accordance with the invention by a process according to claim 1, a formulation according to claim 11, a process according to claim 13 and a dispersion according to claim 15. Advantageous developments are specified in the dependent claims. They may be freely combined unless the opposite is clear from the context.

A process for producing a pharmaceutical formulation in the form of a hybrid system consisting of an amorphous solid solution mixed with crystalline nanoparticles comprises the steps of:

A) providing particles of a polymer, wherein particles of a pharmaceutical active substance are additionally at least partially embedded in the particles of the polymer;

B) heating the particles of the polymer to a predetermined temperature for a predetermined time;

C) cooling the particles of the polymer after the predetermined time to a temperature of 18° C. to 24° C. to produce the pharmaceutical formulation in the form of a hybrid system consisting of an amorphous solid solution mixed with crystalline nanoparticles,

wherein the polymer is at least partially soluble in water and the active substance is at least partially soluble in the polymer,

the particulate pharmaceutical active substance is present in the form of particles having a d₉₀ value in the particle size distribution (volume-based; determined by laser diffraction in accordance with ISO 13320:2009) of ≤1 μm, that the predetermined temperature is within a range from 10 K below the glass transition temperature (determined by DSC in accordance with DIN EN ISO 11357-2 at a heating rate of 10 K/min) of the polymer to the melting temperature of the active substance (determined by DSC in accordance with DIN

EN ISO 11357-2 at a heating rate of 10 K/min), and the total proportion of the active substance in the polymer is greater than the amount of active substance soluble in the polymer at the predetermined temperature.

The present invention provides a pharmaceutical active substance system that comprises both amorphous and nanoparticulate active substance. The particulate fraction is present here in the form of isolated nanoparticles embedded in an amorphous solid solution. This combination of amorphous and nanoparticulate fraction combines the respective properties. In addition, the active substance contents are higher than in a pure amorphous solid solution. There are also advantages in the release kinetics, since not only are there solid active substance nanoparticles present (rate of dissolution adjustable via the particle size), but also an amorphous solid solution having the “spring and parachute” effect typical thereof.

In step A) of the process, polymer particles are provided in which particles of an active substance are at least partially embedded. An equivalent description is that the active substance particles are at least partially encased by the polymer. The polymer is at least partially water-soluble. “Water-soluble” is understood here as meaning that, at 20° C., at least 0.5 g, preferably at least 2 g, of the polymer dissolves in 100 g of water or dissolves with the formation of a gel.

The polymer may be a neutral polymer or a cationic or anionic polyelectrolyte and may be selected from the following group: alkyl celluloses, hydroxyalkyl celluloses, hydroxyalkyl alkyl celluloses, carboxyalkyl celluloses, alkali metal salts of carboxyalkyl celluloses, carboxyalkyl alkyl celluloses, carboxyalkyl cellulose esters, starches, pectins, chitin derivatives, polysaccharides, polyacrylic acid and salts thereof, polymethacrylic acid and salts thereof, polyvinyl alcohol, polyvinylpyrrolidone, polyalkylene oxides, copolymers of the recited polymer types or a mixture of at least two of the abovementioned polymers.

The active substance is at least partially soluble in the polymer. The solubility of the active substance in the polymer at the predetermined temperature is preferably greater than 0.5 g of active substance per 100 g of polymer, more preferably greater than 2 g of active substance per 100 g of polymer. In the case of active substance mixtures, this refers to the most poorly soluble component.

Examples of suitable active substance classes are benzodiazepines, antihypertensives, vitamins, cytostatics, in particular taxol, anaesthetics, neuroleptics, antidepressants, antiviral agents such as anti-HIV agents, antibiotics, antifungals, anti-dementia agents, fungicides, chemotherapy agents, urologics, platelet-aggregation inhibitors, sulfonamides, spasmolytics, hormones, immunoglobulins, sera, thyroid therapeutics, psychotropic agents, antiparkinsonian agents and other antihyperkinetics, ophthalmics, neuropathy products, calcium-metabolism regulators, muscle relaxants, lipid-lowering agents, liver therapeutics, antianginals, cardiac agents, immunotherapeutics, regulatory peptides and inhibitors thereof, hypnotics, sedatives, gynaecological agents, antigout agents, fibrinolytics, enzyme products and transport proteins, enzyme inhibitors, emetics, blood circulation promoters, diuretics, diagnostics, corticosteroids, cholinergics, biliary therapeutics, antiasthmatics, broncholytics, beta-receptor blockers, calcium-channel blockers, ACE inhibitors, anti-arteriosclerosis agents, anti-inflammatories, anticoagulants, antihypotensives, antihypoglycaemics, antihypertensives, antifibrinolytics, antiepileptics, antiemetics, antidotes, antidiabetics, antiarrhythmics, antianaemics, antiallergics, anthelmintics, analgesics, analeptics, aldosterone antagonists, weight-reduction agents or mixtures of at least two of the abovementioned active substance classes.

Active substances that are poorly soluble in water are particularly suitable as active substances. Active substances here are understood as meaning those having a solubility of not more than 1 g, preferably not more than 0.1 g and more preferably not more than 0.01 g, in 100 g of water at 20° C.

As regards the particle size of the active substance, it is preferable that the d₉₀ value of the particle size distribution (d₉₀ in the context of the present invention means that, based on volume, 90% of all particles have a diameter no greater than this value; the determination is carried out by laser diffraction in accordance with ISO 13320:2009) is from ≥10 nm to ≤1 μm, preferably ≥50 nm to ≤500 nm and more preferably ≥30 nm to ≤300 nm. These active substance particles are preferably at least partially crystalline, more preferably crystalline. When this is the case, they may also be described as nanocrystalline active substance particles.

Step B) is a thermal equilibration step comprising heating for a predetermined time to a temperature from 10 K below the glass transition temperature of the polymer to the melting temperature of the active substance. In the case of polymer mixtures, it is the lowest glass transition temperature of the components present that is selected as reference and, in the case of active substance mixtures, it is the lowest melting temperature of the components present. Heating is preferably to ±10 K, more preferably ±5 K of T_{g}, but to no higher than 10 K below the melting temperature of the active substance, since it was surprisingly found that the effect on the formation of the hybrid system consisting of an amorphous solid solution mixed with crystalline nanoparticles is particularly beneficial in this temperature range. Without being bound to any particular theory, it is assumed that the polymer permits increased mobility in the region of T_{g}, with the result that the dissolution properties of the polymer for the active substance, i.e. the embedding of active substance molecules in the polymer matrix, gain in importance.

As already mentioned in the introduction, a process for producing a pharmaceutical formulation is described herein, in which the particles of the polymer are heated to a predetermined temperature for a predetermined time. It is critical that the particles of the polymer themselves reach the predetermined temperature. Consequently, it is, for example, not sufficient for drying during the process to be carried out solely at a certain temperature that is within the temperature range of the predetermined temperature but where the temperature of the polymer particles themselves is not within the temperature range of the predetermined temperature. In order that, after the thermal equilibration step, there is still particulate active substance present and not all the active substance has been dissolved in the polymer, it is envisaged that the total proportion of the active substance in the polymer is greater than the amount of active substance soluble in the polymer at the predetermined temperature. The active substance is preferably dispersed in the polymer up to its thermodynamic saturation, more preferably in the form of a molecular dispersion.

In an embodiment, the predetermined time in step B) is ≥1 second to ≤10 hours. The predetermined time is preferably ≥1 minute to ≤5 hours, more preferably ≥1 hour to ≤4 hours.

In a further embodiment, the material provided in step A) is obtained by milling a suspension comprising particles of the active substance and an aqueous solution of the polymer and then drying. An aqueous solution of the polymer may additionally be used as a means of introducing surfactants, in particular ionic surfactants, into the system.

In a further embodiment, in step B) a suspension comprising particles of the active substance and an aqueous solution of the polymer is atomized from a nozzle of a multi-substance nozzle and a gas having a temperature higher than the predetermined temperature is discharged from another nozzle of the multi-substance nozzle, with the result that the suspension is dried and the dried material is heated to the predetermined temperature. In the production of the hybrid system from the active substance nanosuspension by spray-drying (two-substance nozzle), the spraying of the suspension preferably takes place above the glass transition temperature of the polymer. In an analogous manner to production from the powder, part of the active substance dissolves in the polymer here too, resulting in the formation of an amorphous-crystalline hybrid system. In addition, the crystalline form is maintained.

In a further embodiment, in step B) a suspension comprising particles of the active substance is atomized from a nozzle of a multi-substance nozzle and an aqueous solution of the polymer and of the active substance is atomized from another nozzle of the multi-substance nozzle, resulting in a mixture containing the atomized particle suspension, and additionally a gas having a temperature higher than the predetermined temperature is discharged from another nozzle of the multi-substance nozzle, with the result that the mixture is dried and the dried material is heated to the predetermined temperature. In the production of the hybrid system from the active substance nanosuspension and an active substance-polymer solution by spray-drying (three-substance nozzle), the suspension (preferably aqueous) and solution are sprayed together at temperatures below the glass transition temperature of the polymer. The active substance-polymer solution is produced using solvents suitable for the active substance-polymer system (for example ethanol, acetone). The active substance and additives must be present in completely dissolved form. Present in the solid state is an amorphous solid solution combined with crystalline nanoparticles. In addition, the crystalline form preferably is maintained.

In a further embodiment, the pharmaceutical active substance is selected from: ciclosporin A, ciclosporin G, rapamycin, tacrolimus, deoxyspergualin, mycophenolate mofetil, gusperimus; acetylsalicylic acid, ibuprofen, S(+)-ibuprofen, indometacin, diclofenac, piroxicam, meloxicam, tenoxicam, naproxen, ketoprofen, flurbiprofen, fenoprofen, felbinac, sulindac, etodolac, oxyphenbutazone, phenylbutazone, nabumetone; nifedipine, nitrendipine, nimodipine, nisoldipine, isradipine, felodipine, amlodipine, nilvadipine, lacidipine, benidipine, lercanidipine, furnidipine, niguldipine; α-lipoic acid; muramyl dipeptide or tripeptide, romurtide; vitamin A, D, E or F; vincopectin, vincristine, vinblastine, reserpine, codeine; bromocriptine, dihydroergotamine, dihydroergocristine; chlorambucil, etoposide, teniposide, idoxifene, tallimustine, teloxantrone, tirapazamine, carzelesin, dexniguldipine, intoplicine, idarubicin, miltefosine, trofosfamide, melphalan, lomustine, 4,5-bis(4-fluoroanilino)phthalimide; 4,5-dianilinophthalimide; thymoctonan, prezatide-copper acetate; erythromycin, daunorubicin, gramicidin, doxorubicin, amphotericin B, gentamicin, leucomycin, streptomycin, ganefromycin, rifamexil, ramoplanin, spiramycin; fluconazole, ketoconazole, itraconazole; famotidine, cimetidine, ranitidine, roxatidine, nizatidine, omeprazole; N-[4-methyl-3-(4-pyridin-3-ylpyrimidin-2-ylamino)phenyl]benzamide, N-benzoylstaurosporine; BOC-PhecPhe-Val-Phe-morpholine or the O-[2-(2-methoxyethoxy)acetoxy] derivative thereof; N-[4-(5-cyclopentyloxycarbonylamino-1-methylindol-3-ylmethyl)-3-methoxybenzoyl]-2-vinyloxy]benzenesulfonamide or a mixture of at least two of the abovementioned active substances.

In a further embodiment, the polymer is selected from: methyl cellulose, hydroxymethyl cellulose, hydroxyethyl cellulose, hydroxypropyl cellulose, hydroxybutyl cellulose, hydroxyethyl methylcellulose, hydroxypropyl methylcellulose, carboxymethyl cellulose, sodium carboxymethyl cellulose, carboxymethyl ethyl cellulose, carboxyalkyl cellulose esters, starches, sodium carboxymethyl amylopectin, chitosan, alginic acid, alkali metal salts and ammonium salts of alginic acid, carrageenans, galactomannans, tragacanth, agar-agar, gum arabic, guar gum, xanthan gum, polyacrylic acid and salts thereof, polymethacrylic acid and salts thereof, polyvinyl alcohol, polyvinylpyrrolidone, polyethylene oxide, polypropylene oxide, copolymers of ethylene oxide and propylene oxide, N-vinylpyrrolidone-vinyl acetate copolymers or a mixture of at least two of the abovementioned polymers. Particular preference is given to polyvinylpyrrolidones (in particular K12 and K30 types) and N-vinylpyrrolidone-vinyl acetate copolymers.

In a further embodiment, the particles of the polymer additionally contain an ionic surfactant. The ionic surfactant may be an anionic, cationic or zwitterionic (amphoteric) surfactant. Without being bound to any particular theory, it is assumed that the ionic surfactant in combination with the polymer has a beneficial effect on the stability of the active substance particles during drying. The combination of electrostatic and steric stabilization accordingly makes it possible to redisperse the particles almost completely. It can also be observed that the particles remain in a polymorphic state. This can be documented by X-ray powder diffractometry and by Fourier-transform infrared spectroscopy.

In the polymer/active substance particles in step A), the polymer content may be ≥0.1% to ≤40% by weight and the surfactant content ≥0.001% to ≤10% by weight, in each case based on the total weight of the suspension in step A). A further example of a dosage is a ratio by weight of active substance:polymer:surfactant of ≥0.01 to ≤5:1:≥0.001 to ≤1.

In a further embodiment, the ionic surfactant is selected from:

acylamino acids (and salts thereof), such as: acylglutamates, for example sodium acylglutamate, di-TEA-palmitoyl aspartate and sodium capryl glutamate; acyl peptides, for example palmitoyl-hydrolysed milk protein, sodium cocoyl-hydrolysed soy protein and sodium/potassium cocoyl-hydrolysed collagen; sarcosinates, for example myristoyl sarcosinate, TEA-lauroyl sarcosinate, sodium lauroyl sarcosinate and sodium cocoyl sarcosinate; taurates, for example sodium lauroyl taurate and sodium methyl cocoyl taurate; acyl lactylates, lauroyl lactylate, caproyl lactylate, alaninates; carboxylic acids and derivatives, such as: carboxylic acids, for example lauric acid, aluminium stearate, magnesium alkanolate and zinc undecylenate, ester carboxylic acids, for example calcium stearoyl lactylate and sodium PEG lauramide carboxylate, ether carboxylic acids, for example sodium laureth carboxylate and sodium PEG cocamide carboxylate; phosphoric esters and phosphate salts, such as DEA oleth phosphate and dilaureth phosphate; sulfonic acids and sulfonate salts, such as acyl isethionates, for example sodium/ammonium cocoyl isethionate, alkyl aryl sulfonates, alkyl sulfonates, for example sodium coco monoglyceride sulfate, sodium C-olefin sulfonate, sodium lauryl sulfoacetate and magnesium PEG cocamide sulfate, sulfosuccinates, for example dioctyl sodium sulfosuccinate, disodium laureth sulfosuccinate, disodium lauryl sulfosuccinate and disodium undecylenamido MEA-sulfosuccinate; and also sulfuric esters, such as alkyl ether sulfates, for example sodium laureth sulfate, ammonium laureth sulfate, magnesium laureth sulfate, MIPA laureth sulfate, TIPA laureth sulfate, sodium myreth sulfate and sodium C-pareth sulfate, alkyl sulfates, for example sodium lauryl sulfate, ammonium lauryl sulfate and TEA lauryl sulfate.

In accordance with the invention, ionic surfactant(s) may further be advantageously selected from the group of cationic surfactants. Cationic surfactants that may be used advantageously are alkylamines, alkylimidazoles, ethoxylated amines, quaternary surfactants and esterquats.

Quaternary surfactants contain at least one N atom that is covalently bonded to 4 alkyl or aryl groups. This results in a positive charge, irrespective of pH. Alkyl betaine, alkyl amidopropyl betaine and alkyl amidopropyl hydroxysultaine are advantageous. Cationic surfactants used according to the invention may additionally be preferably selected from the group of quaternary ammonium compounds, in particular benzyltrialkylammonium chlorides or bromides, for example benzyldimethylstearylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltrimethylammonium ether sulfates, alkylpyridinium salts, for example laurylpyridinium or cetylpyridinium chloride, imidazoline derivatives and compounds having a cationic character such as amine oxides, for example alkyldimethylamine oxides or alkylaminoethyldimethylamine oxides. The use of cetyltrimethylammonium salts is particularly advantageous.

In accordance with the invention, ionic surfactant(s) may be advantageously selected from the group of amphoteric surfactants.

Amphoteric surfactants that may be used advantageously are: acylethylenediamines or dialkylethylenediamines, for example sodium acylamphoacetates, disodium acylamphodipropionates, disodium alkylamphodiacetates, sodium acylamphohydroxypropylsulfonates, disodium acylamphodiacetates and sodium acylamphopropionates, and also N-alkylamino acids, for example aminopropylalkylglutamides, alkylaminopropionic acids, sodium alkylimidodipropionates and lauroamphocarboxyglycinate.

Particular preference as surfactant is given to sodium dodecyl sulfate (SDS), sodium docusate, sodium oleate and/or sodium deoxycholate.

In a further embodiment, the active substance and the polymer are present in a relative weight ratio of ≥1:4 to ≤9:1 (preferably ≥1:3 to ≤3:1).

In a further embodiment, the polymer and the surfactant are present in a relative weight ratio of ≥10:1 to ≤300:1 (preferably ≥20:1 to ≤70:1).

A further aspect of the invention is a pharmaceutical formulation comprising a particulate pharmaceutical active substance coated with an at least partially water-soluble polymer, the particulate pharmaceutical active substance being present in the form of particles having a d₉₀ value in the particle size distribution (volume-based; determined by laser diffraction in accordance with ISO 13320:2009) of ≤1 μm, the same active substance additionally being dispersed in the polymer in amorphous form and the total proportion of the active substance in the polymer being greater than the amount of active substance soluble in the polymer at 20° C.

This formulation may be obtained by a process according to the invention. The definitions and embodiments of the process elucidated above are accordingly also applicable to the formulation. The presence of an amorphous dispersion of the active substance in the polymer can be identified by X-ray powder diffractometry (XRPD; Cu-K_{α} radiation) on the basis of the absence of reflections for the crystalline active substance. The embodiment in which the polymer further contains an ionic surfactant merits specific mention.

The invention further relates to a process for producing a suspension of a pharmaceutical formulation, comprising the step of suspending a formulation according to the invention in a suspension medium. The content of active substance after drying may be ≥50% by weight, preferably ≥60% by weight, based on the total weight of the dry substance. The suspension medium is preferably an aqueous suspension medium. It is further preferable that water without further additives is used.

The invention likewise relates to a suspension of a pharmaceutical active substance obtainable by a process according to the invention.

### EXAMPLES

The present invention is elucidated in detail by the examples and figures that follow, but without being restricted thereto. The abbreviation “wt %” means percent by weight and is based on the total weight of the aqueous suspension. PVP K12 is a polyvinylpyrrolidone having a Fikentscher K value (DIN EN ISO 1628-1) of 12. SDS is sodium dodecyl sulfate. KVA 64 is Kollidon® VA64, a vinylpyrrolidone-vinyl acetate copolymer. Instrumental analyses were by Fourier-transform infrared spectroscopy (FTIR) and X-ray powder diffractometry (XRPD).

The glass transition temperatures T_{g} of PVP K12 and KVA 64 were determined by dynamic differential scanning calorimetry (dynamic DSC) in accordance with DIN EN ISO 11357-2 at a heating rate of 10 K/min. T_{g} is 107° C. for PVP K12 and 101° C. for KVA 64.

### Example 1: Indometacin-PVP K12 System

The nanosuspension was prepared using a planetary ball mill (Fritsch Pulverisette 5). For this, 10 wt % of indometacin was stabilized with 6 wt % of PVP K12 and 0.1 wt % of SDS. The polymer-surfactant solutions were prepared and dissolved separately. The solution was then mixed with indometacin powder and the resulting suspension homogenized on a stirring plate. The milling compartments were filled 60% (by volume) with 0.4-0.6 mm milling beads (SiLibeads, zirconium oxide, yttrium-stabilized) and the remaining volume was filled with suspension, taking care to exclude air bubbles. After milling for 1 h 30 min at 400 rpm, a nanosuspension containing particles having a d₉₀<500 nm (Malvern, Mastersizer 2000) was present that could be used for drying.

For freeze-drying, 3 ml vials were filled with 0.7 g of suspension (filling level <1 cm), placed in the freeze-dryer, which was precooled to −40° C., and dried. The resulting powders containing active substance nanoparticles were then used to produce the hybrid systems.

The powders containing active substance nanoparticles (active substance content ≥60 wt % of active substance, PVP K12, SDS) were then baked at approx. 100° C. for up to 4 h. This afforded amorphous-crystalline hybrid systems consisting of both amorphous solid solution and finely dispersed crystalline phase. The mixed systems were demonstrated by XRPD and FTIR measurements.

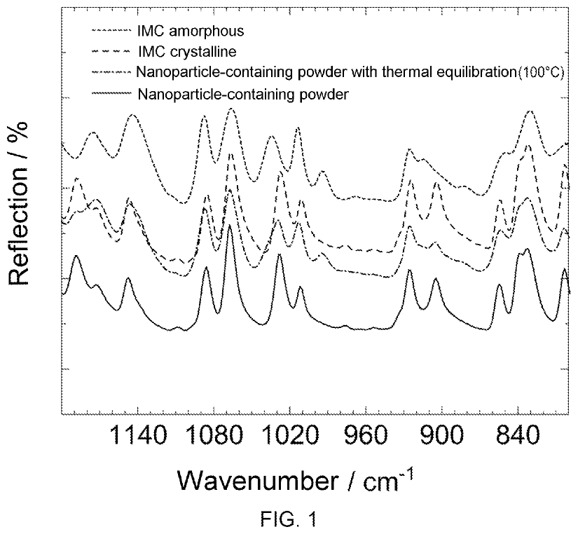

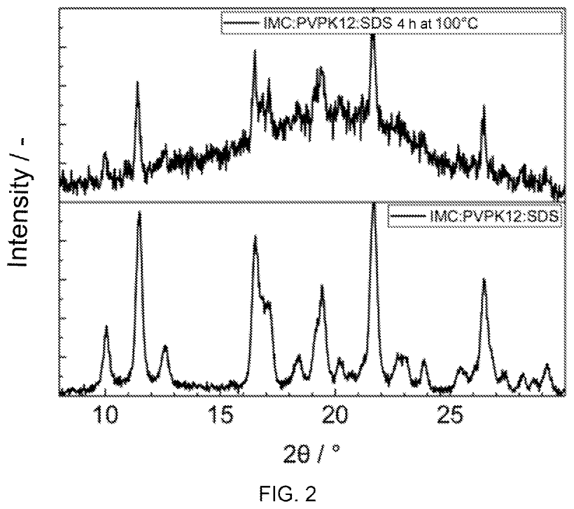

FIG. 1 shows FTIR spectra of amorphous and crystalline indometacin (IMC), of the thermally equilibrated powder containing indometacin nanoparticles (IMC:PVP K12:SDS) and of the powder containing nanoparticles without thermal equilibration. FIG. 2 shows XRP diffraction patterns of thermally equilibrated powder containing indometacin nanoparticles and of the powder containing indometacin nanoparticles without thermal equilibration.

Examination of the FTIR spectra of the purely amorphous and purely crystalline active substance shows clearly that a peak at 994 cm⁻¹ is characteristic of amorphous indometacin and a peak at 904 cm⁻¹ is characteristic of crystalline indometacin.

If the thermally equilibrated sample is compared with the sample without thermal equilibration, it can be seen from FIG. 1 that both amorphous and crystalline fractions are present after thermal equilibration. Moreover, it can be seen from FIG. 2 that both amorphous and crystalline fractions are present in the thermally equilibrated sample, with the crystalline form being unaffected.

### Example 2: Indometacin-KVA 64 System

The powders containing nanoparticles were prepared in an analogous manner to example 1, except that KVA 64 was used instead of PVP K12.

The powders containing active substance nanoparticles (active substance content >60 wt % of active substance, KVA 64 (<40 wt %), SDS) were then baked at approx. 100° C. This afforded amorphous-crystalline hybrid systems consisting of both amorphous solid solution and finely dispersed crystalline phase. The mixed systems were demonstrated by XRPD and FTIR measurements.

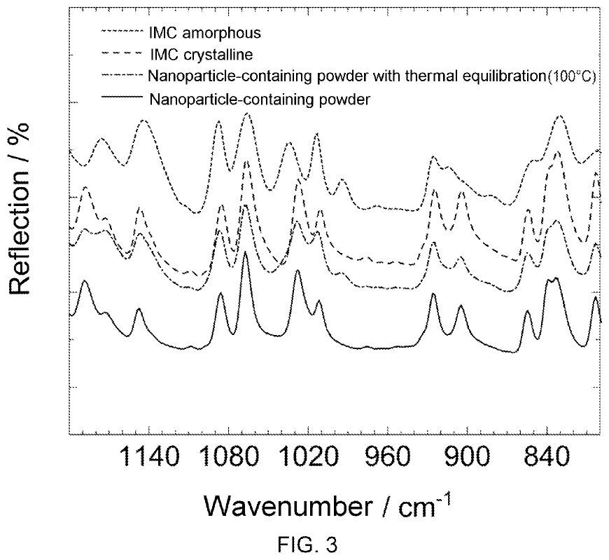

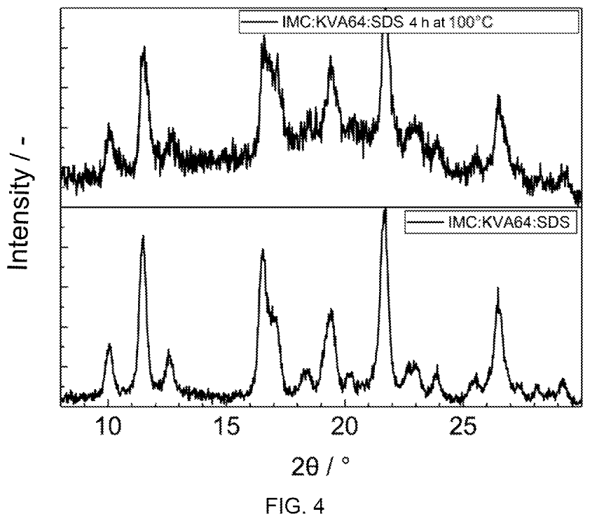

FIG. 3 shows FTIR spectra of amorphous and crystalline indometacin (IMC), of the thermally equilibrated powder containing indometacin nanoparticles (IMC:KVA 64:SDS) and of the powder containing nanoparticles without thermal equilibration. FIG. 4 shows XRP diffraction patterns of thermally equilibrated powder containing indometacin nanoparticles and of the powder containing indometacin nanoparticles without thermal equilibration.

Examination of the FTIR spectra of the purely amorphous and purely crystalline active substance shows clearly that a peak at 994 cm⁻¹ is characteristic of amorphous indometacin and a peak at 904 cm⁻¹ is characteristic of crystalline indometacin. If the thermally equilibrated sample is now compared with the sample without thermal equilibration, it can be seen from FIG. 3 that both amorphous and crystalline fractions are present after thermal equilibration. Moreover, it can be seen from FIG. 4 that both amorphous and crystalline fractions are present in the thermally equilibrated sample, with the crystalline form being unaffected.

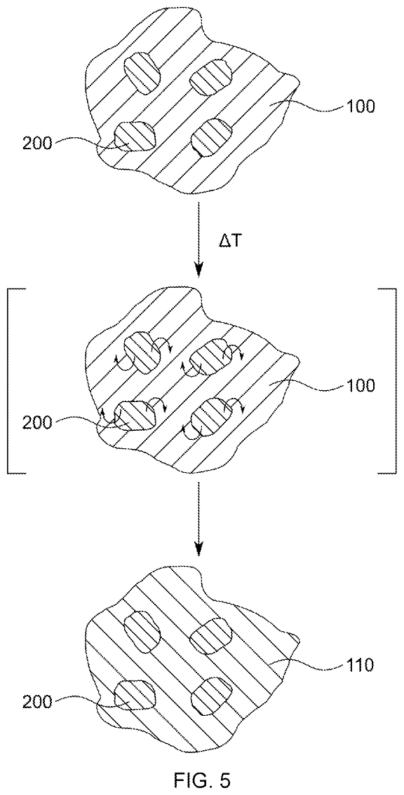

FIG. 5 shows a schematic representation of the process according to the invention, in which particles 100 of an at least partially water-soluble polymer such as PVP K12 or KVA 64 are provided, in which the pharmaceutical active substance is present in the form of a plurality of nanocrystalline particles 200. A suitable active substance is in particular indometacin or naproxen.

An increase in temperature ΔT close to the glass transition temperature T_{g} of the polymer results in partial dissolution of the active substance in the polymer. This is indicated by the curved arrows on the particles 200 in the middle picture.

The bottom picture depicts the pharmaceutical formulation of the invention that is obtainable by the process. In addition to the active substance nanoparticles 200, dissolved active substance is present in the polymer 110. This can be characterized as a monomolecular separation of the active substance or “solid dispersion”.

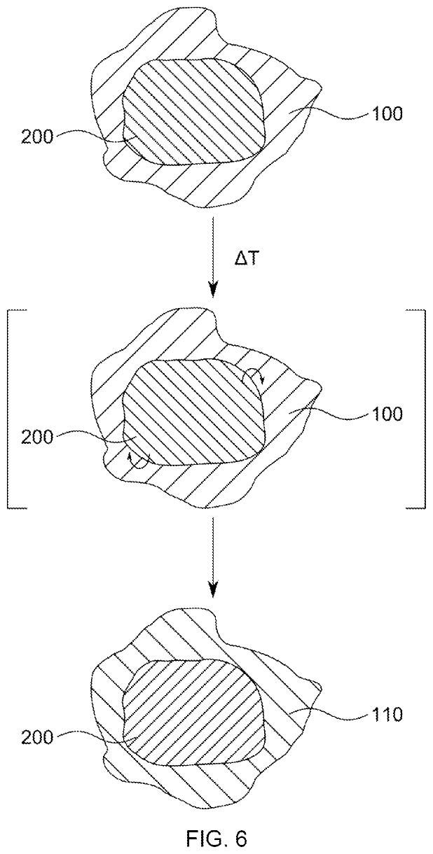

FIG. 6 is a variation on the representation in FIG. 5, in which only a single active substance nanoparticle is present in the polymer 100, 110. The depictions of this in FIGS. 5 and 6 are not necessarily on the same scale.

1. A Process for producing a pharmaceutical formulation in the form of a hybrid system comprising an amorphous solid solution mixed with crystalline nanoparticles,
comprising:
A) providing particles of a polymer, wherein particles of a pharmaceutical active substance are additionally at least partially embedded in the particles of the polymer;
B) heating the particles of the polymer to a predetermined temperature for a predetermined time;
C) cooling the particles of the polymer after the predetermined time to a temperature of 18° C. to 24° C. to produce the pharmaceutical formulation in the form of a hybrid system comprising an amorphous solid solution mixed with crystalline nanoparticles,
wherein the polymer is at least partially soluble in water and the active substance is at least partially soluble in the polymer,
wherein
the particulate pharmaceutical active substance is present in the form of particles having a d₉₀ value in the particle size distribution (volume-based; determined by laser diffraction in accordance with ISO 13320:2009) of ≤1 μm,
the predetermined temperature is within a range from 10 K below the glass transition temperature (determined by DSC in accordance with DIN EN ISO 11357-2 at a heating rate of 10 K/min) of the polymer to the melting temperature of the active substance (determined by DSC in accordance with DIN EN ISO 11357-2 at a heating rate of 10 K/min), and the total proportion of the active substance in the polymer is greater than the amount of active substance soluble in the polymer at the predetermined temperature.

2. Process according to claim 1, wherein the predetermined temperature is within a range of ±10 K of the glass transition temperature (determined by DSC in accordance with DIN EN ISO 11357-2 at a heating rate of 10 K/min) of the polymer to no higher than 10 K below the melting temperature of the active substance (determined by DSC in accordance with DIN EN ISO 11357-2 at a heating rate of 10 K/min).

3. Process according to claim 1, wherein the predetermined time in B) is ≥1 second to 10 hours.

4. Process according to claim 1, wherein the material provided in A) is obtained by milling a suspension comprising particles of the active substance and an aqueous solution of the polymer and then drying.

5. Process according to claim 1, wherein, in B), a suspension comprising particles of the active substance and an aqueous solution of the polymer is atomized from a nozzle of a multi-substance nozzle and a gas having a temperature higher than the predetermined temperature is discharged from another nozzle of the multi-substance nozzle, with the result that the suspension is dried and the dried material is heated to the predetermined temperature.

6. Process according to claim 1, wherein, in B), a suspension comprising particles of the active substance is atomized from a nozzle of a multi-substance nozzle and an aqueous solution of the polymer and of the active substance is atomized from another nozzle of the multi-substance nozzle, with the result that a mixture containing the atomized particle suspension arises, and additionally a gas having a temperature higher than the predetermined temperature is discharged from another nozzle of the multi-substance nozzle, with the result that the mixture is dried and the dried material is heated to the predetermined temperature.

7. Process according to claim 1, wherein the pharmaceutical active substance is selected from: ciclosporin A, ciclosporin G, rapamycin, tacrolimus, deoxyspergualin, mycophenolate mofetil, gusperimus; acetylsalicylic acid, ibuprofen, S(+)-ibuprofen, indometacin, diclofenac, piroxicam, meloxicam, tenoxicam, naproxen, ketoprofen, flurbiprofen, fenoprofen, felbinac, sulindac, etodolac, oxyphenbutazone, phenylbutazone, nabumetone; nifedipine, nitrendipine, nimodipine, nisoldipine, isradipine, felodipine, amlodipine, nilvadipine, lacidipine, benidipine, lercanidipine, furnidipine, niguldipine; α-lipoic acid; muramyl dipeptide or tripeptide, romurtide; vitamin A, D, E or F; vincopectin, vincristine, vinblastine, reserpine, codeine; bromocriptine, dihydroergotamine, dihydroergocristine; chlorambucil, etoposide, teniposide, idoxifene, tallimustine, teloxantrone, tirapazamine, carzelesin, dexniguldipine, intoplicine, idarubicin, miltefosine, trofosfamide, melphalan, lomustine, 4,5-bis(4-fluoroanilino)phthalimide; 4,5-dianilinophthalimide; thymoctonan, prezatide-copper acetate; erythromycin, daunorubicin, gramicidin, doxorubicin, amphotericin B, gentamicin, leucomycin, streptomycin, ganefromycin, rifamexil, ramoplanin, spiramycin; fluconazole, ketoconazole, itraconazole; famotidine, cimetidine, ranitidine, roxatidine, nizatidine, omeprazole; N-[4-methyl-3-(4-pyridin-3-ylpyrimidin-2-ylamino)phenyl]benzamide, N-benzoylstaurosporine; BOC-PhecPhe-Val-Phe-morpholine or the O-[2-(2-methoxyethoxy)acetoxy] derivative thereof; N-[4-(5-cyclopentyloxycarbonylamino-1-methylindol-3-ylmethyl)-3-methoxybenzoyl]-2-vinyloxy]benzenesulfonamide or a mixture thereof.

8. Process according to claim 1, wherein the polymer is selected from methyl cellulose, hydroxymethyl cellulose, hydroxyethyl cellulose, hydroxypropyl cellulose, hydroxybutyl cellulose, hydroxyethyl methylcellulose, hydroxypropyl methylcellulose, carboxymethyl cellulose, sodium carboxymethyl cellulose, carboxymethyl ethyl cellulose, carboxyalkyl cellulose esters, starches, sodium carboxymethyl amylopectin, chitosan, alginic acid, alkali metal salts and ammonium salts of alginic acid, carrageenans, galactomannans, tragacanth, agar-agar, gum arabic, guar gum, xanthan gum, polyacrylic acid and salts thereof, polymethacrylic acid and salts thereof, polyvinyl alcohol, polyvinylpyrrolidone, polyethylene oxide, polypropylene oxide, copolymers of ethylene oxide and propylene oxide, N-vinylpyrrolidone-vinyl acetate copolymers or a mixture thereof.

9. Process according to claim 1, wherein the particles of the polymer additionally comprise an ionic surfactant.

10. Process according to claim 1, wherein the active substance and the polymer are present in a relative weight ratio of 1:4 to 9:1.

11. Process according to claim 8, wherein the polymer and the surfactant are present in a relative weight ratio of 10:1 to 300:1.

12. Pharmaceutical formulation comprising a particulate pharmaceutical active substance coated with an at least partially water-soluble polymer,
wherein
the particulate pharmaceutical active substance is present in the form of particles having a d₉₀ value in the particle size distribution of ≤1 μm,
in the polymer, the same active substance is additionally also dispersed in amorphous form and
the total proportion of the active substance in the polymer is greater than the amount of active substance soluble in the polymer at 20° C.

13. Formulation according to claim 12, wherein the polymer additionally comprises an ionic surfactant.

14. Process for producing a suspension of a pharmaceutical formulation comprising suspending a formulation according to claim 12 in a suspension medium.

15. Suspension of a pharmaceutical active substance obtainable by a process according to claim 14.
